(19) United States
(12) Patent Application Publication
(10) Pub. No.: US 2021/0193324 A1
(43) Pub. Date: Jun. 24, 2021

(54) EVALUATING PATIENT RISK USING AN ADJUSTABLE WEIGHTING PARAMETER

(71) Applicant: KONINKLIJKE PHILIPS N.V., EINDHOVEN (NL)

(72) Inventors: WILLIAM ANTHONY TRUSCHEL, MONROEVILLE, PA (US); ALBERTUS CORNELIS DEN BRINKER, EINDHOVEN (NL); IGOR BEREZHNYY, EINDHOVEN (NL)

(21) Appl. No.: 17/120,587

(22) Filed: Dec. 14, 2020

Related U.S. Application Data
(60) Provisional application No. 62/950,695, filed on Dec. 19, 2019.

(51) Int. Cl. G16H 50/30; G16H 10/60; G06F 17/15

(52) U.S. Cl. CPC G16H 50/30; G06F 17/153; G16H 10/60

(57) ABSTRACT

The present disclosure describes a system for providing model-based assessments of patient risk, including a processor configured to obtain, for each of a plurality of patients, a symptomatic risk score and a demographic risk score, obtain a weighting coefficient for each patient's respective demographic risk score, multiply each weighting coefficient by the respective demographic risk scores to produce respective a weighted demographic risk score for each patient, obtain, a three dimensional weighting parameter surface in which one dimension is symptomatic risk score, one dimension is demographic risk score, and one dimension is weighting parameter such that for each combination of symptomatic risk score and demographic risk score, a weighting parameter is defined, generate a two dimensional array of patient scores according to their respective symptomatic risk score and their respective weighted symptomatic risk score, and output the stratified two dimensional array of patient risk coordinates to a display.

### CROSS-REFERENCE TO RELATED APPLICATIONS

This patent application claims the priority benefit under 35 U.S.C. § 119(e) of U.S. Provisional Application No. 62/950,695, filed on Dec. 19, 2019, the contents of which are herein incorporated by reference.

### BACKGROUND OF THE INVENTION

### 1. Field of the Invention

The present disclosure pertains to a system and method for providing improved model-based predictions of patient risk combining longitudinal real time symptom tracking with demographic information. The system and method uses a weighting parameter that changes the risk assessment according to a polynomial surface of both symptoms and demographics.

### 2. Description of the Related Art

Patient risk stratification makes use of historical patient health records, statistical models, various sensors and patient monitors, as well as non-clinical and environmental factors. Stratification can allow for care decisions to be made in a manner that can result in increased health care efficiency, potential cost savings, and reduction in hospitalizations.

Decision support models in health care may be used for determining recommended treatments for a patient. The treatment suggested by a decision support model may optimize survival, quality of life, cost-effectiveness, or a combination thereof. Although automated and other computer-assisted treatment recommendation systems exist, such systems may often disregard treatment consequences extending beyond physical health states and including mental, emotional, or social functioning. For example, while current treatment recommendation systems may define the concept of quality of life (e.g., via questionnaires), the use of this concept in practice may be limited to population measures. On the individual level, these concepts may not be concrete and detailed enough to be used to determine the best care path for the patient that incorporates the affective impact of a treatment on the patient's life. These and other drawbacks exist.

### SUMMARY OF THE INVENTION

Accordingly, one or more aspects of the present disclosure relate to a system for providing model-based assessments of patient risk, the system including a processor configured by machine-readable instructions to obtain, for each of a plurality of patients, a symptomatic risk score and a demographic risk score, obtain a weighting coefficient for each patient's respective demographic risk score, multiply each weighting coefficient by the respective demographic risk scores to produce respective a weighted demographic risk score for each patient, obtain, a three dimensional weighting parameter surface in which one dimension is symptomatic risk score, one dimension is demographic risk score, and one dimension is weighting parameter such that for each combination of symptomatic risk score and demographic risk score, a weighting parameter is defined, generate a two dimensional array of patient scores according to their respective symptomatic risk score and their respective weighted symptomatic risk score, and output the stratified two dimensional array of patient risk coordinates to a display.

An aspect of some embodiments relates to such a system wherein each symptomatic risk score is generated based on measured values of a plurality of symptoms and/or each demographic risk score is generated based on measured values of a plurality of demographic characteristics.

An aspect of some embodiments relates to such a system wherein the measured values are each respectively weighted based on a plurality of predetermined correlation coefficients.

An aspect of some embodiments includes generating each symptomatic risk score based on a model using the plurality of symptoms as an input and/or each demographic risk score is generated based on a model using the plurality of demographic characteristics as an input.

An aspect of some embodiments includes such a system wherein the processor is further configured to obtain the weighting coefficient for each patient's respective demographic risk score by generating, at a user interface, a slider configured to allow the user to select a variation from the demographic risk score based on patient-specific information known to the user.

An aspect of some embodiments includes such a system wherein the variation is selected based on one or more factors selected from the group consisting of: social determinants of health, patient compliance, provider engagement, and/or statistics pertaining locally to the health facility providing care.

One or more aspects includes a method for providing model-based assessments of patient risk comprising, obtaining, for each of a plurality of patients, a symptomatic risk score and a demographic risk score, obtaining a weighting coefficient for each patient's respective demographic risk score, multiplying each weighting coefficient by the respective demographic risk scores to produce respective a weighted demographic risk score for each patient, obtaining a three dimensional weighting parameter surface in which one dimension is symptomatic risk score, one dimension is demographic risk score, and one dimension is weighting parameter such that for each combination of symptomatic risk score and demographic risk score, a weighting parameter is defined, generating a two dimensional array of patient scores according to their respective symptomatic risk score and their respective weighted symptomatic risk score, and outputting the stratified two dimensional array of patient risk coordinates to a display.

An aspect of one or more embodiments includes a system for providing model-based assessments of patient risk, the system comprising, means for obtaining, for each of a plurality of patients, a symptomatic risk score and a demographic risk score, means for obtaining a weighting coefficient for each patient's respective demographic risk score, means for multiplying each weighting coefficient by the respective demographic risk scores to produce respective a weighted demographic risk score for each patient, means for obtaining, a three dimensional weighting parameter surface in which one dimension is symptomatic risk score, one dimension is demographic risk score, and one dimension is weighting parameter such that for each combination of symptomatic risk score and demographic risk score, a weighting parameter is defined, means for generating a two dimensional array of patient scores according to their respective symptomatic risk score and their respective weighted symptomatic risk score, and means for displaying the stratified two dimensional array of patient risk coordinates.

An aspect of some embodiments includes a machine readable tangible medium programmed to cause a processor to perform the steps of one or more of the foregoing methods, or to control the operation of one or more of the foregoing systems.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system configured for providing model-based predictions of quality of life implications of a treatment via individual-specific machine learning models;

FIG. 2 is a graph illustrating a relationship between probability of exacerbation vs. number of exacerbations in the previous year, and including seasonal variation in probability;

FIG. 3 illustrates seasonal variation in number of admissions and readmission rates;

FIG. 4 shows an example of a set of demographic risk correlation coefficients for COPD patients;

FIG. 5 shows an example of a set of symptomatic risk correlation coefficients for COPD patients;

FIG. 6 is an example of a three dimensional weighting parameter surface generated based on demographic risk scores and symptomatic risk scores, in accordance with one or more embodiments;

FIG. 7A is an example of a distribution of patients plotted according to weighted symptomatic risk and demographic risk; FIG. 7B the same patients with their distribution modified by application of a stratification process in accordance with one or more embodiments;

FIGS. 8 and 9 illustrate a fit between the model and expert training data;

FIG. 10 illustrates an example of an adjustment to weighting of parameters by a user;

FIG. 11 illustrates an example of a stratified patient group in accordance with one or more embodiments; and

FIG. 12 is a flow chart illustrating steps of a method in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

In an embodiment, a system 10 is configured to perform patient-risk stratification is designed for managing patients with chronic obstructive pulmonary disease (COPD).

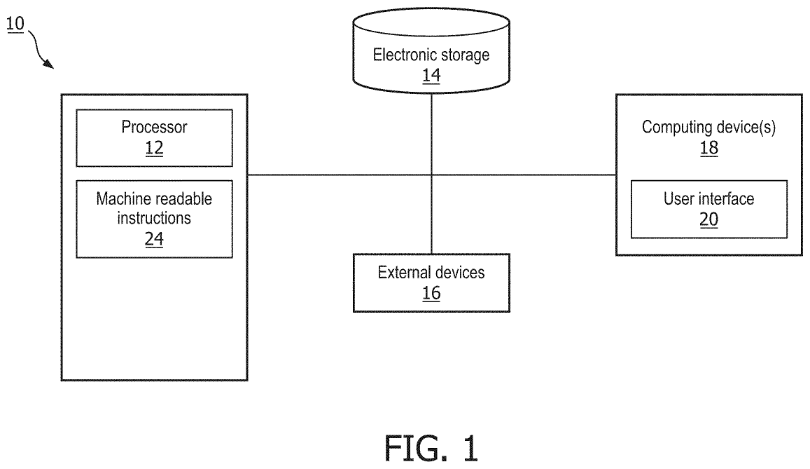

FIG. 1 is a schematic illustration of a system 10 configured for providing performing patient risk stratification using symptomatic risk factors and demographic risk factors.

In some embodiments, system 10 comprises processors 12, electronic storage 14, external devices 16, computing device 18 having a user interface 20, or other components. The external devices 16 may be, for example, hospital equipment that obtains data on various health states of a patient.

Electronic storage 14 comprises electronic storage media that electronically stores information (e.g., health, demographic, social information associated with individual patients. The electronic storage media of electronic storage 14 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 14 may be (in whole or in part) a separate component within system 10, or electronic storage 14 may be provided (in whole or in part) integrally with one or more other components of system 10 (e.g., computing device 18, etc.). In some embodiments, electronic storage 14 may be located in a server together with processors 12, in a server that is remote from a caregiving location. Electronic storage 14 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 14 may store software algorithms, information determined by processors 12, information received via processors 12 and/or graphical user interface 20 and/or other external computing systems, information received from external devices 16, and/or other information that enables system 10 to function as described herein.

External devices 16 include sources of information and/or other resources. For example, external devices 16 may include a population's electronic medical record (EMR), the population's electronic health record (EHR), or other information. In some embodiments, external devices 16 include health information related to the population. In some embodiments, the health information comprises demographic information, vital signs information, medical condition information indicating medical conditions experienced by individuals in the population, treatment information indicating treatments received by the individuals, care management information, and/or other health information. In some embodiments, external devices 16 include sources of information such as databases, websites, etc., external entities participating with system 10 (e.g., a medical records system of a health care provider that stores medical history information of patients, publicly and privately accessible social media websites), one or more servers outside of system 10, and/or other sources of information. In some embodiments, external resources 16 include components that facilitate communication of information such as a network (e.g., the internet), electronic storage, equipment related to Wi-Fi technology, equipment related to Bluetooth® technology, data entry devices, sensors, scanners, and/or other resources. In some embodiments, some or all of the functionality attributed herein to external resources 16 may be provided by resources included in system 10.

Processors 12, electronic storage 14, external devices 16, computing device 18, and/or other components of system 10 may be configured to communicate with one another, via wired and/or wireless connections, via a network (e.g., a local area network and/or the internet), via cellular technology, via Wi-Fi technology, and/or via other resources. It will be appreciated that this is not intended to be limiting, and that the scope of this disclosure includes embodiments in which these components may be operatively linked via some other communication media. In some embodiments, processors 12, electronic storage 14, external devices 16, computing device 18, and/or other components of system 10 may be configured to communicate with one another according to a client/server architecture, a peer-to-peer architecture, and/or other architectures.

In embodiments, external devices 16 may include, for example, home monitoring equipment that is either fixed or mobile. It may include a wearable or other user device that monitors heart rate, oxygen saturation, blood pressure, temperature, or other information relevant to the user's health or well-being.

Computing device 18 may be configured to provide an interface between one or more users, and system 10. In some embodiments, computing device 18 is and/or is included in desktop computers, laptop computers, tablet computers, smartphones, smart wearable devices including augmented reality devices (e.g., Google Glass), wrist-worn devices (e.g., Apple Watch), and/or other computing devices associated with a user. In some embodiments, computing device 18 facilitates presentation of a list of individuals assigned to a care manager, or other information. Accordingly, computing device 18 comprises a user interface 20. Examples of interface devices suitable for inclusion in user interface 20 include a touch screen, a keypad, touch sensitive or physical buttons, switches, a keyboard, knobs, levers, a camera, a display, speakers, a microphone, an indicator light, an audible alarm, a printer, tactile haptic feedback device, or other interface devices. The present disclosure also contemplates that computing device 18 includes a removable storage interface. In this example, information may be loaded into computing device 18 from removable storage (e.g., a smart card, a flash drive, a removable disk, etc.) that enables caregivers or other users to customize the implementation of computing device 18. Other exemplary input devices and techniques adapted for use with computing device 18 or the user interface include an RS-232 port, RF link, an IR link, a modem (telephone, cable, etc.), or other devices or techniques.

Processor 12 is configured to provide information processing capabilities in system 10. As such, processor 12 may comprise one or more of a digital processor, an analog processor, a digital circuit designed to process information, an analog circuit designed to process information, a state machine, or other mechanisms for electronically processing information. Although processor 12 is shown in FIG. 1 as a single entity, this is for illustrative purposes only. In some embodiments, processor 12 may comprise a plurality of processing units. These processing units may be physically located within the same device (e.g., a server), or processor 12 may represent processing functionality of a plurality of devices operating in coordination (e.g., one or more servers, computing device, devices that are part of external resources 16, electronic storage 14, or other devices.)

As shown in FIG. 1, processor 12 is configured via machine-readable instructions 24 to execute one or more computer program components. The computer program components may comprise one or components for performing steps of a method in accordance with one or more embodiments. Processor 12 may be configured to execute components by software; hardware; firmware; some combination of software, hardware, or firmware; or other mechanisms for configuring processing capabilities on processor 12.

It should be appreciated that although the components are illustrated in FIG. 1 as being co-located within a single processing unit, in embodiments in which processor 12 comprises multiple processing units, one or more components may be located remotely from the other components. The description of the functionality provided by the different components described below is for illustrative purposes, and is not intended to be limiting, as any of components may provide more or less functionality than is described. For example, one or more of the components may be eliminated, and some or all of its functionality may be provided by other components. As another example, processor 12 may be configured to execute one or more additional components that may perform some or all of the functionality attributed below to one of components.

In an embodiment, an intelligent system for modeling and predicting patient risk is applicable to patients with chronic obstructive pulmonary disease (COPD). The system may be used, for example, to apply stratigraphic triage for patients based on the symptomatic and environmental data collected from sensors in the home, hospital, or other patient locations. An algorithm for the model may assign a weighted risk score that prioritizes patients based on aggregate combinations of symptomatic data multiplied by a weighting parameter. Proper stratification of patients may enable a provider to assign resources to patients who are most in need, patients who are most likely to improve, or other health outcome goals. For example, it may be possible to identify timely low-cost interventions in order to reduce hospitalizations or serious exacerbations. Moreover, an intelligent system may allow scaling of managed care to a wider and larger population.

In an embodiment, the demographic risk is based on a case worker's assessment of a probability that the patient will need an emergency hospitalization within a number of months, for example, 24 months, and takes the form of a demographic risk score. In an embodiment, the risk score is selected from a range from 0 to 100, but in principle any range could be used, for example 0-1 or 1-10. Though the examples discussed herein relate to hospitalization risk, in principle the method could be applied to any type of outcome. For example, COPD exacerbation. In principle, a set of parameters and weightings relating to risk of cancer relapse could be used with cancer patients, while parameters and weightings relating to risk of heart disease progression or heart attack could be used with cardiology patients. The use of input from a case worker may allow for someone who, for example, knows the patient personally, knows the status of the local healthcare system intimately, or other additional information not captured in the statistical models, can estimate a probability that the specific individual patient may be sensitive to risk. For example, they may factor in the patient/doctor relationship or the patient personality.

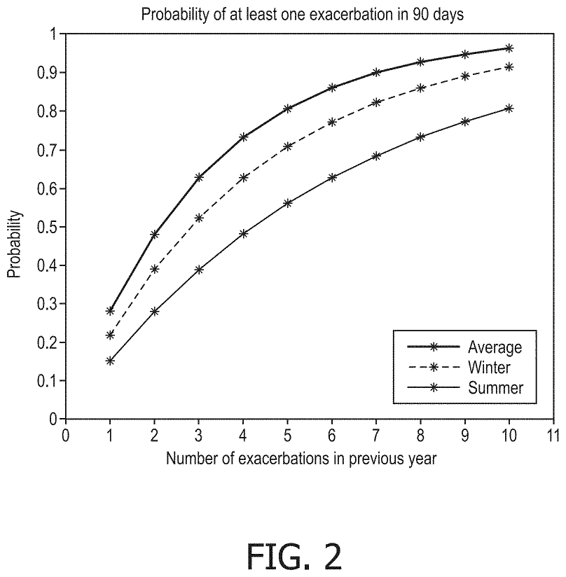

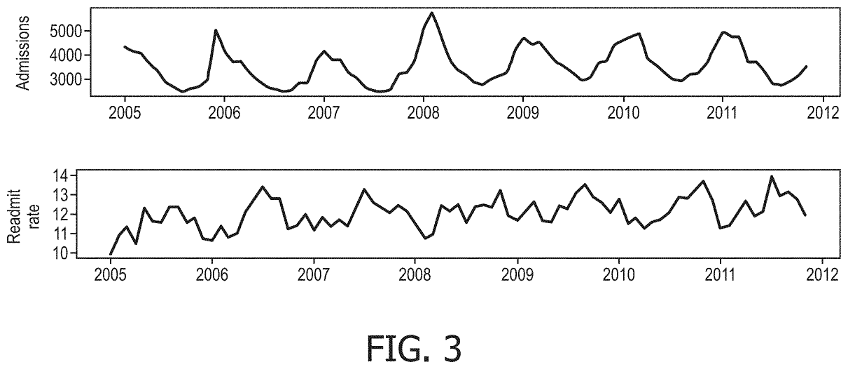

In an embodiment, the demographic is based on a model built from demographic information related to how likely a patient of a particular phenotype is likely to have an acute exacerbation within the next 90 days. For example, it is known from a study containing 16,565 patients in the UK [Kerkhof, M, et al, Predicting frequent COPD exacerbations using primary care data. 2015 Int. J. Chron. Obstruct. Pumon. Dis., 10: 2439-2450], that the number of exacerbations recorded in the previous twelve months is the most associative predictor of future exacerbations as illustrated in FIG. 2. A model would then estimate the probability of exacerbations within the next 90 days based on this history, for example assigning a demographic risk score of 50 for patients with 3 exacerbations in the previous year, while assigning a demographic risk score of 20 for patients with only 1 exacerbation in the previous year. Furthermore, this score may be adjusted according to seasonal effects that are known to increase the probability of a patient having an emergency admission as illustrated in FIG. 3. For example, a study of over 300,000 admissions to a hospital shows a strong seasonal effect to the probability of readmission in COPD patients due to influenza and rhinovirus pandemics. [Simmering J E, et al, Identifying patients with COPD at high risk of readmission, 2016 Int. J. Chron. Obstruct. Pumon. Dis., 3(4): 729-738].

The demographic risk score could therefore be adjusted according to FIG. 2 from a combined analysis of previous exacerbations and seasonal probabilities.

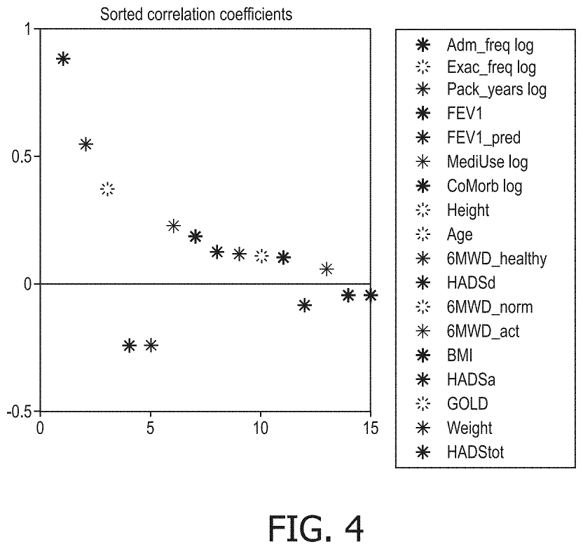

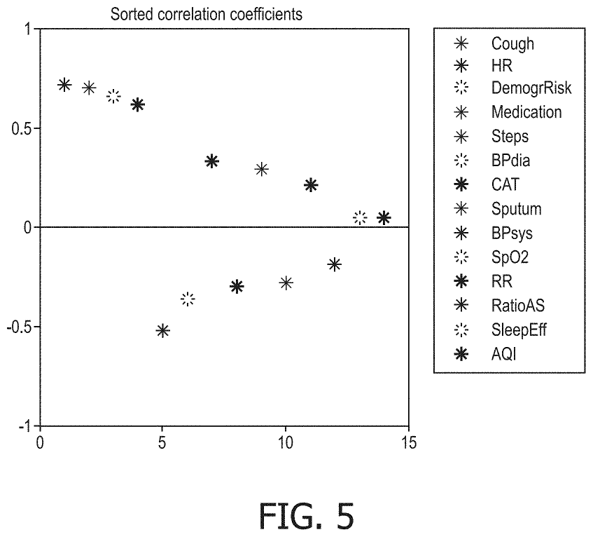

The case worker's risk score assignment may be guided by a recommendation based on a machine learning model. Such machine learning models may be built by observing expert processing of data relating to a training set of patients with multiple parameters. The particular machine learning model used may vary, but the model may be based on a behavioral clone of the case worker himself or other users of the system as the machine learns common behaviors and trends. The machine learning model may also be based on reinforced learning correlating risk in the practice to previous examples of readmission or exacerbation using the demographics data. In an embodiment, there may be tens of parameters, for example, more than 20 parameters, that are known to correlate to likelihood of re-hospitalization. FIGS. 4 and 5 each illustrate lists of parameters that are known to correlate to hospitalization in COPD patients. In an embodiment, the case worker is able to, based on knowledge of the patient and the state of the local healthcare system, adjust the score.

That is, in an embodiment of a system for risk stratification, the primary load of estimating risk is performed by the artificial intelligence system analyzing data from patient monitors, however, the non-clinical, personalized, social, economic, psychological, and historical information collected by those who manage an individual patient can be incorporated.

In an embodiment, therefore, a manually chosen, but machine-guided assignment of a demographic risk score is produced. This score provides a baseline assessment of the patient's overall risk or mortality in order to adjust the sensitivity of the statistical algorithms that are analyzing the patient symptoms. Guided manual entry of the demographic risk score may be done by a case manager who is enrolling a patient in the system. The demographic risk score may then be combined with a symptomatic risk score to provide a weighted symptomatic risk score that will be used to stratify the population of patients.

The weighting parameter is assigned based on a surface that relates weighting parameter to demographic risk score chosen by the case manager as aided by the model's output (for example as a number between 0-100) along with a symptomatic risk score based on sensor and environmental data which may likewise be a score between 0 and 100. The algorithm then computes a weighted symptomatic score by multiplying the symptomatic risk score by the weighting parameter described by the surface.

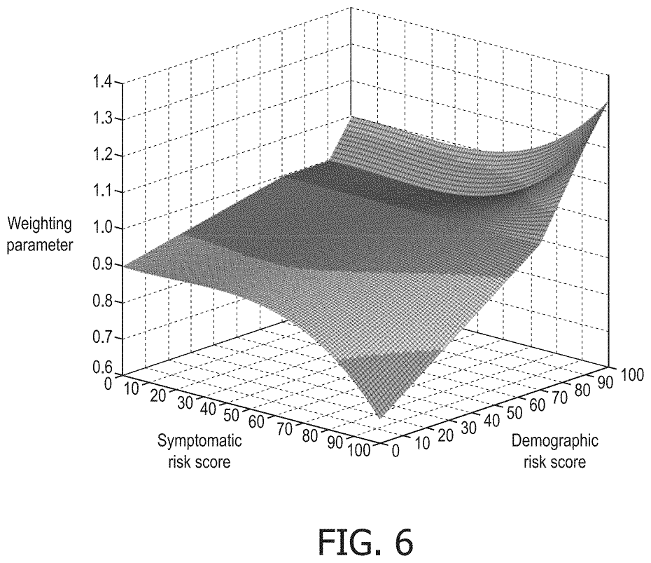

An example of a surface of this type is illustrated in FIG. 6. In the surface of FIG. 4, portions that are on the higher end of both risk scores are amplified. Moreover, for any portion of the surface, portions on the higher end of demographic risk score are generally amplified relative to portions on the lower end of demographic risk score. Portions on the lower end of demographic risk score and higher end of symptomatic risk score are attenuated the most. In one interpretation, an edge along zero symptomatic risk rises slowly from a low attenuation at a demographic risk score of zero to a moderate amplification at a demographic risk score of 100. A corresponding edge along the 100 symptomatic risk score begins at a point of greatest attenuation and rises more quickly to a point of greatest amplification. A surface stretching between these two edges is interpolated resulting in a curve that is generally flat in the middle and steeper on the edges, with a steeper edge along the highest risk dimension.

In practice, the surface itself can be generated empirically, using existing data, and observing the population shift from a random distribution to a prioritized distribution. In the case where population data is historical, and subsequent instances of patient outcomes is known, various optimization/fitting operations can be performed to determine what surface best matches past performance. In this regard, and AI training method or a human-guided training can be performed on the generated surface to obtain a surface that is expected to best model the behavior of the populations under treatment.

In effect, much of the input in the model may be subjective. For example, the symptom, “sputum,” from FIG. 5 is generally a subjective measure collected from patient input in which the patient simply selects a value from 1-5 for a given day to characterize the sputum that is being produced. As a result, the necessary weighting surface will likewise tend to involve some subjective modification in response to empirical results. If, for example, a risk score of 71 is determined to be high risk, and a very high percentage of patients are being assigned a risk of 71 or above, then it may be determined that the system is not providing sufficiently accurate predictions and resources are not being properly allocated. In this event, the surface may be adjusted such that risk scores are appropriately shifted.

As may be seen in FIG. 6, the surface has a highest degree of steepness closer to the lowest demographic risk score and closest to the highest demographic risk score. This corresponds to a high gain for values that are associated with patients who are at a highest risk, and a high attenuation for values associated with patients who are at a lowest risk. That is, the system is particularly sensitive to patients who are high risk such that a change in symptoms will tend to trigger a higher priority intervention. Patients who are low risk, on the other hand, have a steep attenuation so that even if symptoms deviate somewhat, the clinician is not triggered to perform some type of intervention. A polynomial surface in this embodiment was designed to produce the desired effect described above.

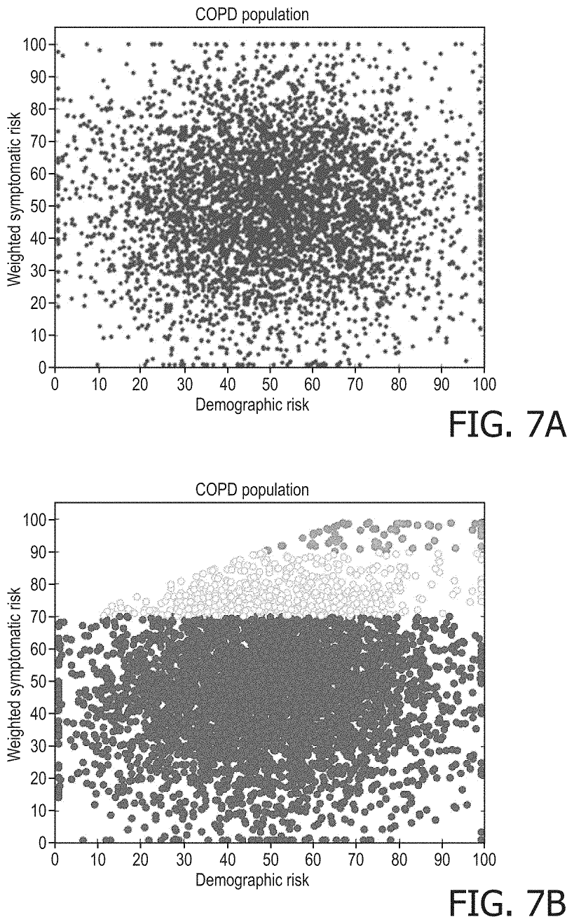

An example of the results of an embodiment of this process is shown in FIGS. 7A and 7B. In FIG. 7A, the population is shown having a distribution that is approximately a normal distribution. FIG. 7B then shows the result of multiplying the normal distribution of 7A by the weighting surface, wherein clusters of patients with similar risks, or who would be more likely to need intervention, are more easily identified. That is, in the normal distribution, it can be difficult to distinguish risk among those members of the patient population who have a high demographic risk or a high symptomatic risk, but once the weighting has been applied, it becomes more clear which patients need an intervention. The weighting parameter will increase the weighted score for symptomatic patients that have had for example, multiple exacerbations in the previous year, live in an area of an influenza pandemic or have been designated by the case worker has a high risk patient by associated socio-economic such as disengagement with the health care provider.

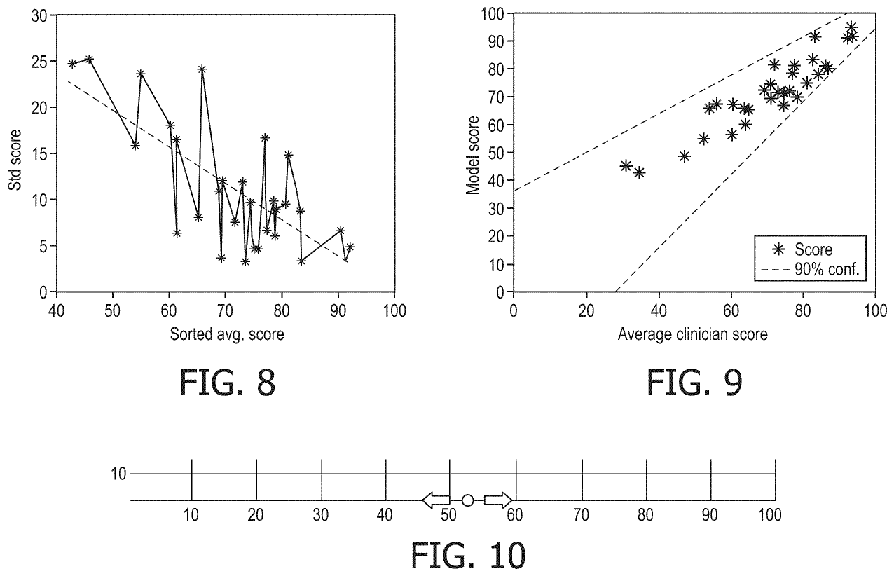

In an example of generating a set of demographic risk values for COPD, demographic risk annotations produced by expert evaluators were analyzed and a regression model was built. The sorted average score was plotted against the standard score as shown in FIG. 8.

Because uncertainty decreased with higher risk as seen in FIG. 9, the numerical data scale was modified to produce a uniform uncertainty across the risk scale before regression was computed. Further observation of the correlation coefficients suggested a model order of no greater than 5 was necessary to achieve adequate performance. Joint optimization resulted in the use of the top 5 predictive parameters: Admission frequency, exacerbation frequency, forced expiration volume vs % predicted, number of comorbidities and number of medications prescribed. The correlation coefficients for the top parameters are shown in FIG. 8.

In an embodiment, symptomatic risk is determined based on measurements of a number of symptoms related to the disease under evaluation. As noted above, FIG. 5 illustrates a number of such symptoms. Specifically, cough, heart rate, demographic information, medication, steps, diastolic blood pressure, COPD assessment test (CAT), sputum, systolic blood pressure, oxygen saturation (SPO₂), respiratory rate, ratio of night activity to day activity (AS Ratio), sleep efficiency, and air quality index may all be measured at varying intervals in the patient's location.

The component of demographic information involved in determining the symptomatic risk is different from demographic risk as discussed below. Instead, what this means is that some degree of demographic information is take into account when determining a symptomatic risk. For example, an infant's symptomatic risk differs from an adult's with respect to elevated temperatures. Similarly, a low oxygen saturation represents a higher symptomatic risk for a one lunged patient than for a two lunged patient.

These symptoms may be measured, for example, using a wearable or local monitoring device such as a watch that measures pulse, a pulse oximeter that the user places on a finger, a blood pressure cuff, or the like. These devices collectively or alone may comprise all or part of the external devices 16 described above.

In an embodiment, the external devices 16 may include a home health monitoring device, smartwatch, wearable biosensors, a stand-alone unit with which a patient may input information, or an application run on a PC, smart device, tablet, mobile phone, or other device. For example, a smart device or tablet may include one or more applications configured to administer the COPD assessment test, which is a questionnaire that addresses certain COPD symptoms and asks the patient to self-assess on a number of symptom questions relating to cough, phlegm, chest tightness, ability to conduct activities, sleep, and overall energy, for example. As can be seen in FIG. 5, some of the questions addressed in the CAT have a greater correlation to risk than the test as a whole, while others have lower correlation to risk.

Input on symptoms may be collected daily, or at a different interval. As will be appreciated, measures like heart rate and oxygen saturation, while they may be measured more or less continuously, will tend to vary over the course of a day, so an average may be preferable to any instantaneous measurement. Likewise, a measure like steps (per day) or ratio of day to night activities make sense primarily in a day to day value rather than as an instant value. Something like sleep efficiency may incorporate a several day window moving average, or the like. The selected interval for measurements can affect the results, so consistency should be maintained both over a patient monitoring period, and between the collection of training data and monitoring of patient populations. To the extent that there is inconsistency, the weighting function may require modification in response.

Demographic risk takes into account patient demographic data and does not have a symptomatic component. That is, while symptomatic risk includes patient demographic information, demographic risk does not directly take into account patient symptom information.

In an example, as shown in FIG. 4, demographic information relevant to COPD demographic risk may include admission frequency, exacerbation frequency, smoking pack years, forced expiratory volume in one second (FEV1), predicted FEV1, frequency of medicine use, co-morbidity (i.e., presence of additional disease), height, age, 6MWD (six minute walk distance—normal for age and gender and actual result from test), hospital anxiety and depression scale (HADS) wherein HADS includes two separate components for anxiety and depression (HADSa) and (HADSd) and the total is HADStot, body mass index (BMI), GOLD (Global Initiative for COPD, a qualitative rating of the degree of patient's disease), weight.

These factors may be measured from time to time, or for factors like height, may be considered to be constant. For varying measures like weight and FEV1, self-assessments may be performed by the patient, and/or periodic or daily measurements may be performed by a caregiver. HADS, for example, may be measured by a questionnaire provided on a user device similar to the CAT described above. In a typical setting, the patient could be asked to respond to both questionnaires in a single interaction with a home health monitoring system. FEV1 may be measured on a home spirometer, or less frequent measurements may be used based on tests administered by a medical professional.

In practice, the enrolled patient is assigned a value and placed on the demographic risk axis based on the demographic information in accordance with the model. However, this value does not take into account any social factors and therefore may not represent a good estimate of that patient's actual risk.

The case manager then, knowing social factors that are relevant to the risk for that patient, may apply an adjustment taking social factors into account. Additionally, the case manager may take into account the patient's personality. For example, to the extent that a case manager is aware of a particular patient's compliance in taking medicine or performing other self-care activities. For example, such self-care might include engaging in physical or mental therapy, exercising, attending follow-up visits, smoking cessation, diet improvement, or the like.

In an embodiment, the case manager is provided a demographic risk value by the system, and then is presented with a slider of the type illustrated in FIG. 10 by which he or she may adjust the base value. This adjustment, in addition to taking into account such patient-specific social determinants of health may take into account items like provider engagement. For example, to the extent a case manager is aware that a patient's doctor is highly engaged with that patient (or with all patients in general) then a downward risk adjustment might be made. Furthermore, local statistics may be taken into account, particularly if the underlying model is based on widely gathered statistics. That is, statistics that pertain to the particular facility providing health care to the patient may be more relevant than global statistics used to derive the model.

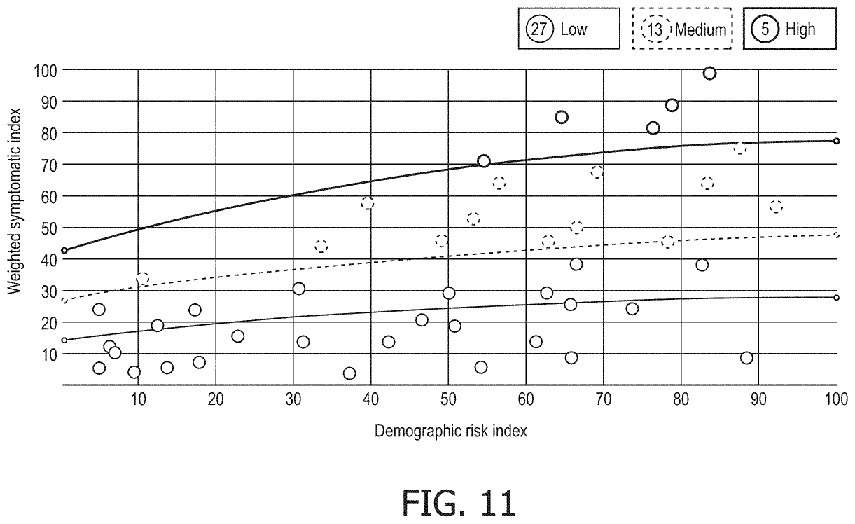

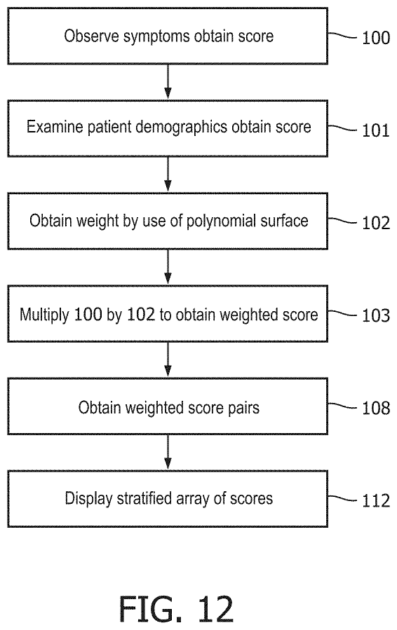

FIG. 11 is an example of a set of patient data that has been stratified in accordance with an embodiment. As may be observed, certain of the patients who have apparently high demographic risk, are nevertheless of low overall risk. The high risk patients are well clustered at the upper right, and decisions to attend to those patients could be made based on this.

A method in accordance with an embodiment is illustrated in FIG. 10. The method begins by first obtaining 100, for each of a plurality of patients, a symptomatic risk score. As described above, the symptomatic risk score may be derived from a plurality of items of from sensors and surveys monitoring the patient and having been processed or analyzed by a mathematical model, for example a regression of order 5 (i.e., a regression performed on the five most well-correlated categories of data) relating symptoms to risk.

The method proceeds by obtaining 101 a demographic risk score for each patient. In an embodiment, the score may be obtained from an operator. As described above, the demographic risk score relates patient-specific risk based on patient history, social determinants of health, provider engagement, and/or local statistics.

The system then obtains a weighting coefficient with the use of the polynomial surface and multiplies the symptomatic score by the weighting parameter produce respective a weighted symptomatic risk score 103 for each patient. The demographic risk score, 101 and the weighted symptomatic risk score 103 are combined in a pair 108. The weights should be chosen to produce weighted scores that remain between 0-100 (or whatever the scale is that is being used for the risk scores). The resultant pairs, 108, are then displayed for the case worker to triage a population of patients.

A three dimensional weighting parameter surface is obtained in which one dimension is symptomatic risk score, one dimension is demographic risk score, and one dimension is weighting parameter such that for each combination of symptomatic risk score and demographic risk score, a weighting parameter is defined. This surface can be replaced with a table of values that produces the same effect by assigning greater weight to higher symptomatic risk scores in proportional to demographic risk.

A two dimensional array of patient scores are obtained 108 (i.e., a pair of scores for each patient) according to their respective symptomatic risk score and their respective weighted symptomatic risk score. The array produces a stratified two dimensional array of patient risk coordinates.

The stratified two dimensional array of patient risk coordinates are displayed 112 on a display for use by a care coordinator to determine priority patients, and to order interventions in accordance with the determined priority. An example of such an array as displayed is illustrated in FIG. 11.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A system for providing model-based assessments of patient risk, the system comprising:
a processor configured by machine-readable instructions to:
obtain, for each of a plurality of patients, a symptomatic risk score and a demographic risk score;
obtain a weighting coefficient for each patient's respective demographic risk score;
multiply each weighting coefficient by the respective demographic risk scores to produce respective a weighted demographic risk score for each patient;
obtain, a three dimensional weighting parameter surface in which one dimension is symptomatic risk score, one dimension is demographic risk score, and one dimension is weighting parameter such that for each combination of symptomatic risk score and demographic risk score, a weighting parameter is defined;
generate a two dimensional array of patient scores according to their respective symptomatic risk score and their respective weighted symptomatic risk score; and
output the stratified two dimensional array of patient risk coordinates to a display.

2. The system of claim 1, wherein each symptomatic risk score is generated based on measured values of a plurality of symptoms and/or each demographic risk score is generated based on measured values of a plurality of demographic characteristics.

3. The system of claim 2, wherein the measured values are each respectively weighted based on a plurality of predetermined correlation coefficients.

4. The system of claim 2, wherein each symptomatic risk score is further generated based on a model using the plurality of symptoms as an input and/or each demographic risk score is generated based on a model using the plurality of demographic characteristics as an input.

5. The system of claim 1, wherein the processor is further configured to obtain the weighting coefficient for each patient's respective demographic risk score by generating, at a user interface, a slider configured to allow the user to select a variation from the demographic risk score based on patient-specific information known to the user.

6. The system of claim 5, wherein the variation is selected based on one or more factors selected from the group consisting of: social determinants of health, patient compliance, provider engagement, and/or statistics pertaining locally to the health facility providing care.

7. A method for providing model-based assessments of patient risk comprising:
obtaining, for each of a plurality of patients, a symptomatic risk score and a demographic risk score;
obtaining a weighting coefficient for each patient's respective demographic risk score;
multiplying each weighting coefficient by the respective demographic risk scores to produce respective a weighted demographic risk score for each patient;
obtaining a three dimensional weighting parameter surface in which one dimension is symptomatic risk score, one dimension is demographic risk score, and one dimension is weighting parameter such that for each combination of symptomatic risk score and demographic risk score, a weighting parameter is defined;
generating a two dimensional array of patient scores according to their respective symptomatic risk score and their respective weighted symptomatic risk score; and
outputting the stratified two dimensional array of patient risk coordinates to a display.

8. The method of claim 7, wherein each symptomatic risk score is generated based on measured values of a plurality of symptoms and/or each demographic risk score is generated based on measured values of a plurality of demographic characteristics.

9. The method of claim 8, wherein the measured values are each respectively weighted based on a plurality of predetermined correlation coefficients.

10. The method of claim 8, wherein each symptomatic risk score is further generated based on a model using the plurality of symptoms as an input and/or each demographic risk score is generated based on a model using the plurality of demographic characteristics as an input.

11. The method of claim 7, further comprising generating, at a user interface, a slider configured to allow the user to select a variation from the demographic risk score based on patient-specific information known to the user and wherein the variation is used to obtain the weighting coefficient for each patient's respective demographic risk score.

12. The method of claim 11, wherein the variation is selected based on one or more factors selected from the group consisting of: social determinants of health, patient compliance, provider engagement, and/or statistics pertaining locally to the health facility providing care.

13. A system for providing model-based assessments of patient risk, the system comprising:
means for obtaining, for each of a plurality of patients, a symptomatic risk score and a demographic risk score;
means for obtaining a weighting coefficient for each patient's respective demographic risk score;
means for multiplying each weighting coefficient by the respective demographic risk scores to produce respective a weighted demographic risk score for each patient;
means for obtaining, a three dimensional weighting parameter surface in which one dimension is symptomatic risk score, one dimension is demographic risk score, and one dimension is weighting parameter such that for each combination of symptomatic risk score and demographic risk score, a weighting parameter is defined;
means for generating a two dimensional array of patient scores according to their respective symptomatic risk score and their respective weighted symptomatic risk score; and
means for displaying the stratified two dimensional array of patient risk coordinates.
